(19) United States
(12) Patent Application Publication
(10) Pub. No.: US 2025/0041823 A1
(43) Pub. Date: Feb. 6, 2025

(54) PURIFICATION OF ORGANIC SOLVENTS

(71) Applicant: Dow Global Technologies LLC, Midland, MI (US)

(72) Inventors: Linping Qian, Shanghai (CN); Gengfeng Zheng, Shanghai (CN); Qi Jiang, Shanghai (CN); Qihao Wang, Shanghai (CN)

(73) Assignee: Dow Chemical (China) Investment Company Limited, Shanghai (CN)

(21) Appl. No.: 18/718,879

(22) PCT Filed: Dec. 14, 2021
(86) PCT No.: PCT/CN2021/137785

§ 371 Date: Jun. 12, 2024

(51) Int. Cl. B01J 20/16; B01J 20/28; B01J 20/30

(52) U.S. Cl. CPC B01J 20/165; B01J 20/28011; B01J 20/28045; B01J 20/28064; B01J 20/28066; B01J 20/2808; B01J 20/3078; B01J 20/3085

(57) ABSTRACT

A modified zeolite material used for removing metal impurities from an organic solvent composition; the modified zeolite material comprising (i) a hydrogen-based zeolite having hydrogen functionalities, (ii) an ammonium-based zeolite having ammonium functionalities, or (iii) a combination of a hydrogen-based zeolite having hydrogen functionalities and an ammonium-based zeolite having ammonium functionalities; the zeolite material containing a pore size of from 3 Å to 20 Å; and the zeolite material selectively removing metal impurities present in the solvent, to an impurity content of less than 1 ppb; a process for preparing the above modified zeolite material; and a process for removing contaminants from organic solvents using the above modified zeolite material.

### FIELD

The present invention relates to modified zeolites, processes for purifying organic solvents by removing contaminants from the organic solvents, processes for purifying the organic solvents by removing metal contaminants using modified zeolites, and purified organic solvents.

### BACKGROUND

Organic solvents are widely used in polymer cleaning, dissolution and stripping applications. For certain applications, such as electronics processing, pharma, daily products, and the like, use of a pure solvent, that is, a solvent which is free of metal and other contaminants, is required by the users in such industries. For example, solvents (e.g., electronics processing chemicals) used in the manufacture of electronic materials, must contain extremely a low (e.g., less than 10 ppb) concentration of impurities.

Heretofore, distillation methods or processes using multiple purification steps have been implemented for purifying chemicals (solvents) and providing an electronic grade standard product. The reason being is that most single-step purification processes, other than distillation, can only selectively remove one target impurity. Such single purification technologies cannot meet the strict quality requirements of the electronics industry for purifying solvents that are used in electronic processing.

What is needed in the industry is a less complicated and less costly method of purifying organic solvents different than a distillation method. In particular, it is desirable to purify organic solvents such as glycol ethers and glycol ether acetates, used in applications such as electronics processing where the amount of metal and other contaminants present in the organic solvent is strictly controlled to a very low maximum level. Therefore, an effective and efficient way of removing metal impurities from an organic solvent is highly desirable.

Some known processes for removing various metal contaminants from a solution or aqueous medium employ materials such as: a silicate composition as mentioned in WO2021091816A1; a zeolite type material as mentioned in WO2021032754A1, JP03770538B2, JP2006052118A, JP2004250259A, CN111807635A, and CN108033456B); a porous material incorporated with an oxygen-containing compound of iron, copper, aluminum, titanium, and/or zirconium as mentioned in U.S. Pat. No. 7,429,551B2; or a molecular sieve in a packed bed as mentioned in CN109476582A0. However, it would be desirable to have an effective and efficient method for removing metals present in organic solvents.

### SUMMARY

In one embodiment, the present invention is directed to a modified zeolite for removing metal impurities to less than 1 ppb of heavy metals and anions, from an organic solvent composition containing metal impurities greater than 1 ppb; the modified zeolite comprising (i) a hydrogen-based zeolite having hydrogen functionalities, (ii) an ammonium-based zeolite having ammonium functionalities, or (iii) a mixture of such hydrogen-based zeolite and ammonium-based zeolite; wherein the zeolite contains an average pore size sufficient to adsorb metal impurities and other contaminants. For example, the average pore size of the modified zeolite can be from 3 Å to 20 Å in one general embodiment to selectively adsorb metal impurities and water present in the organic solvent to provide a purified organic solvent.

In another embodiment, the present invention is directed to a purification process for removing contaminants from organic solvents using the above modified zeolite. For example, in one preferred embodiment, the process of the present invention includes removing metal contaminants and non-metallic ionic contaminants from an organic solvent or a mixture of organic solvents using the above modified zeolite. For example, in another preferred embodiment, the present invention relates to a purification process including using the above modified zeolite in a fixed bed with high metal removal efficiency (to ppt metal level) and water removal in organic solvents. For example, in still another preferred embodiment, the present invention is directed to the purification of organic solvents such as glycol ethers and glycol ether acetates. In yet another preferred embodiment, the present invention relates to a process for removing metal contaminants and cation contaminants from an organic solvent or from a mixture of two or more organic solvents including the steps of: (a) preparing a fixed bed of the above modified zeolite absorbent material; and (b) contacting an organic solvent or a mixture of organic solvents with the fixed bed of modified zeolite absorbent material of step (a).

Generally, the metal contaminants present in the organic solvent can be, for example, Na, K, Ca, Al, Fe, Ni, Zn, Cu, Cr and Sn; and the initial concentration of each of the metal contaminants in the organic solvent can be 0.1 ppb or greater. Therefore, one objective of some embodiments of the present invention is to provide a more efficient process than known processes wherein the process can be used to remove a high level of one or more metal contaminants. For example, an objective of some embodiments of the present invention is to provide a process to achieve a purified organic solvent having a metal contaminant level of less than 50 ppt.

Various embodiments of the present invention are described in more detail in the following Detailed Description.

### DETAILED DESCRIPTION

Specific embodiments of the present application are described herein below.

Unless stated to the contrary or otherwise, implicit from the context, or customary in the art, all percentages, parts, ratios, and the like amounts, are defined by, or based on, weight. parts and percent values are based on weight. For example, all percentages stated herein are weight percentages (wt %), unless otherwise indicated. And, all test methods disclosed herein are current as of the filing date of this disclosure.

All temperatures used herein are in degrees Celsius (° C.).

“Room temperature (RT)” and “ambient temperature” herein means a temperature between 20° C. and 26° C., unless specified otherwise.

The term “composition,” as used herein, refers to a mixture of materials which comprises the composition, as well as reaction products and decomposition products formed from the materials of the composition.

The term “zeolite”, as used herein, means microporous, aluminosilicate minerals commonly used as commercial absorbents.

The term “modified zeolite”, as used herein, means a conventional zeolite which has been treated to remove alkali or alkaline metals present in the conventional zeolite.

The terms “comprising,” “including,” “having,” and their derivatives, are not intended to exclude the presence of any additional component, step or procedure, whether or not the same is specifically disclosed. In order to avoid any doubt, all compositions claimed through use of the term “comprising” may include any additional additive, adjuvant, or compound, whether polymeric or otherwise, unless stated to the contrary. In contrast, the term “consisting essentially of” excludes from the scope of any succeeding recitation any other component, step, or procedure, excepting those that are not essential to operability. The term “consisting of” excludes any component, step, or procedure not specifically delineated or listed. The term “or,” unless stated otherwise, refers to the listed members individually as well as in any combination. Use of the singular includes use of the plural and vice versa.

The numerical ranges disclosed herein include all values from, and including, the lower and upper value. For ranges containing explicit values (e.g., a range from 1, or 2, or 3 to 5, or 6, or 7), any subrange between any two explicit values is included (e.g., the range 1 to 7 above includes subranges 1 to 2; 2 to 6; 5 to 7; 3 to 7; 5 to 6; and the like.).

As used throughout this specification, the abbreviations given below have the following meanings, unless the context clearly indicates otherwise: “=” means “equal(s)” or “equal to”; “<” means “less than”; “>” means “greater than”; “≤” means “less than or equal to”; ≥” means “greater than or equal to”; “@” means “at”; ppm=parts per million; ppb=parts per billion; ppt=parts per trillion; BV/hr=bed volume/hour(s); “MT”=metric ton(s); g=gram(s); mg=milligram(s); Kg=kilogram(s); L=liters; g/L=gram(s) per liter; μL=microliter(s); “g/cm³” or “g/cc”=gram(s) per cubic centimeter; g/10 min=gram(s) per 10 minutes; mg/mL=milligrams per milliliter; “kg/m³=kilogram(s) per cubic meter; ppm=parts per million by weight; pbw=parts by weight; rpm=revolutions per minute; m=meter(s); mm=millimeter(s); cm=centimeter(s); μm=micron(s) or micrometer(s); nm=nanometer(s); Å=angstrom(s); min=minute(s); s=second(s); ms=millisecond(s); hr=hour(s); Pa=pascals; MPa=megapascals; Pa-s=Pascal second(s); mPa-s=millipascal second(s); g/mol=gram(s) per mole(s); g/eq=gram(s) per equivalent(s); Mn=number average molecular weight; Mw=weight average molecular weight; pts=part(s) by weight; 1/s or sec-1=reciprocal second(s) [s⁻¹]; ° C.=degree(s) Celsius; ° C./min=degree(s) Celsius per minute; psi=pounds per square inch; kPa=kilopascal(s); %=percent; vol %=volume percent; mol %=mole percent; and wt %=weight percent.

In general, the absorbent material of the present invention is a modified zeolite including the following matrix:

M₂O·xAl₂O₃·ySiO₂·zH₂O

where in the above matrix, M is at least one of hydrogen (H); ammonium (NH₄); NRH₃, NR₂H₂, NR₃H, NR₄ and R is methyl; and metal oxides including, for example, Li₂O, Na₂O, K₂O, and mixtures thereof. In other embodiments, the M₂O constituent of the molecular structure M₂O·xAl₂O₃·ySiO₂·zH₂O can be M′O wherein M′ comprises divalent metals such as MgO, CaO, SrO, BaO; and mixtures thereof. In the above matrix, x, y and z are mole ratios and x is from 0.2 to 5 in one embodiment, from 0.5 to 3 in another embodiment; and from 0.8 to 1.5 in still another embodiment. In the above matrix, y is from 1 to 25 in one embodiment, from 1.5 to 10 in another embodiment; and from 2 to 5 in still another embodiment. In the above matrix, z is from 1 to 20 in one embodiment, from 2 to 10 in another embodiment; and from 3 to 6 in still another embodiment.

In other embodiments, the modified zeolite has an average pore size of from 3 Å to 20 Å (0.3 nm to 2.0 nm) in one general embodiment; from 3 Å to 15 Å in another embodiment; from 3 Å to 11 Å in still another embodiment; from 3 Å to 10 Å in even still another embodiment; and from 4 Å to 10 Å in yet another embodiment.

In other embodiments, the modified zeolite has a specific surface area of >500 m²/g in one general embodiment, from 500 m²/g to 5,000 m²/g in another embodiment; and from 1,000 m²/g to 2,000 m²/g in still another embodiment.

In some embodiments, the modified zeolite of the present invention having the molecular structure of M₂O·xAl₂O₃·ySiO₂·zH₂O described above has a cation exchange capacity of 0.1 eq/mol to 1.5 eq/mol in one embodiment, from 0.5 eq/mol to 1.4 eq/mol in another embodiment; and from 1.0 eq/mol to 1.3 eq/mol in still another embodiment.

The modified zeolite of the present invention also is stable in liquids having a pH value of from 0 to 12 in one embodiment, from 3 to 10 in another embodiment; and from 5 to 8 in still another embodiment. By “stable” in liquids, with reference to the modified zeolite of the present invention, it is meant that the elements (e.g., metals) of the zeolite do not leach out/release into a liquid (e.g., a solvent) and the zeolite keeps its integrity in liquids that have a pH of from 0 to 12. By “unstable” in liquids, with reference to the modified zeolite of the present invention, it is meant that the elements (e.g., metals) of the zeolite will leach out into the liquid (at a pH of 0-12); and therefore, for example, the leached metal will add to the metal contaminant content of the liquid. An unstable zeolite is undesired since its effect would be to increase the metal contamination content of the solvent which is the opposite effect desired by using the modified zeolite of the present invention.

The shape of the modified zeolite is not critical and can be in the shape of, for example, beads, bars, cubes, powders, other irregular shapes, and mixtures thereof.

In general, the modified zeolite can include A, X, Y, USY, and STI types. In a preferred embodiment, the modified zeolite includes A and Y types.

In another embodiment, the composition of the modified zeolite can include, for example, a composition comprising a combination of the following matrixes:
(a) M₂O·xAl₂O₃·ySiO₂·zH₂O (where M=hydrogen); (b) M′₂O·xAl₂O₃·ySiO₂·zH₂O (where M′=ammonium); and (c) optionally, M″20 xAl₂O₃·ySiO₂·zH₂O (where M″=a metal oxide).

In a preferred embodiment, the modified zeolite composition is a combination of component (a) and (b) having a weight ratio a:b of from 1:99 to 99:1.

When the optional component (c) is used, the weight ratio (mixture of components (a) and (b)):c is from 1:1 to 99:1 in one embodiment. In a preferred embodiment, when the optional component (c) is used in preparing the modified zeolite composition, the components (a)-(c) are mixed in accordance with the following process steps: step (I) provide component (c); step (II) mix component (a) and (b); and step (III) mix component (c) with the mixture of component (a) or (b).

In a broad embodiment, a process for preparing the modified zeolite useful in the present invention includes, for example, the following steps of:
Step (I): placing an unmodified (conventional) zeolite material in a fixed bed column;
Step (II): treating the unmodified (conventional) zeolite material of step (I) by flushing the non-modified zeolite material in the fixed bed column of step (I) with a weak acid (for hydrogen-based zeolite), a weak base/ammonia salt (for ammonium-based zeolite); or a mixture thereof (for a mixture of hydrogen-based zeolite and ammonium-based zeolite); and
Step (III): activating the zeolite material from step (II) by calcinating the zeolite material from step (II) at a dehydration temperature of from 200° C. to 400° C. to provide dehydrated zeolite material.

The weak acids used in step (II) can include, for example, acetic acid, propanoic acid, phosphoric acid; tartaric acid; and mixtures thereof. The weak bases used in step (II) can include, for example, ammonia, ammonia salts, and mixtures thereof. The ammonia salts used in step (II) to treat the unmodified zeolite material to prepare a modified zeolite, can include, for example, (NH₄)₂SO₄; CH₃COONH₄; CH₃COON(CH₃)₄; [N(CH₃)]₄Cl; and mixtures thereof.

The flow rate of the flushing step (II) can be, for example, from 0.1 BV/hr to 20 BV/hr (BV=bed volume) in one general embodiment; from 0.5 BV/hr to 10 BV/hr in another embodiment; and from 1 BV/hr to 5 BV/hr in still another embodiment.

The temperature of the flushing step (II) can be, for example, from room temperature to up to 100° C. in one general embodiment; from 40° C. to 80° C. in another embodiment; and from 50° C. to 70° C. in still another embodiment.

The temperature of the activation step (III) can be, for example, from 200° C. to 400° C. in one general embodiment; from 220° C. to 350° C. in another embodiment; and from 250° C. to 300° C. in still another embodiment.

In carrying out the above process, an optional inert gas padding may be used, if desired. For example, the inert gas can be nitrogen, argon, and mixtures thereof.

The process embodiments of the present invention described above for preparing a modified zeolite composition, and the steps thereof as described above, can be carried out by conventional equipment known to those skilled in the art. For example, the flushing step (II) to form the hydrogen-based zeolite and/or the ammonium-based zeolite material can be carried out by known fixed bed components and the heating step (III) can be carried out by heaters commonly known in the art.

Some advantageous properties and/or benefits exhibited by the modified zeolite material used for the purification of solvents process of the present invention can include, for example: (1) using ammonia-based chemistry effectively replaces alkali or alkaline metals presented in the zeolite; (2) a lower concentration of metal leaching occurs with the use of the modified zeolite material of the present invention, for example, the amount of leaching is maintained at a level of from 1 ppb to less than 1 ppm in one general embodiment; (3) a reduction of alkali in the zeolite material prior to using the zeolite material to purify a solvent can be achieved, for example, the concentration of the alkali can be from 0.01 wt % to <0.1 wt % in one general embodiment; and (4) after modification of the zeolite material, the zeolite material provides a solvent with an increase in purity to as low as ppt levels, for example, <1,000 ppt in one general embodiment.

Once the modified zeolite material useful in the present invention is prepared as described above, the zeolite material is placed in a column and subjected to dehydration in accordance with the following steps of:
Step (1): placing the modified zeolite material, prepared by steps of the above zeolite preparation process, in a column;
Step (2): filling the column with inert gas such as nitrogen or argon; at a gas pressure of from 0.1 MPa to 0.3 MPa in one general embodiment; and
Step (3): heating the column, with the zeolite material and the solvent present in the column, to a temperature of from room temperature to 100° C. in one general embodiment for a period of time of from 10 min to 1,000 min in one general embodiment to form a dehydrated zeolite material.

After the modified zeolite material useful in the present invention is dehydrated as described above, a solvent to be purified is passed through the dehydrated zeolite material in the column and subjected to metal exchange. The metal exchange process can occur after the dehydration steps (1)-(3) above in accordance with the following additional steps of:
Step (4): continue passing a solvent to be purified through the hydrated zeolite material bed in the column at a flow rate of from 0.1 BV/hr to 20 BV/hr in one general embodiment; and
Step (5): heating the column, with the hydrated zeolite material and the solvent present in the column, to a temperature of from 200° C. to 400° C. in one general embodiment for a period of time from 60 min to 300 min in one general embodiment.

After the solvent is passed through modified zeolite material to activate the metal exchange process as described above, the modified zeolite material may be re-used for processing another solvent to be purified by regenerating the zeolite material in the column prior to passing the solvent through the zeolite material. In general, the regeneration process is carried out using the same zeolite modification process (steps (I)-(III)) described above.

In general, the process of the present invention is applicable to purifying organic solvents; and particularly for purifying a single organic solvent and/or a mixture of two or more organic solvents. For example, the organic solvents to be purified may include the following: dipropylene glycol methyl ether, tri propylene glycol methyl ether, propylene glycol ethyl ether, propylene glycol propyl ether, ethylene glycol propyl ether, ethylene glycol butyl ether, diethylene glycol methyl ether, diethylene glycol ethyl ether, diethylene glycol propyl ether, diethylene glycol butyl ether, ethanol, isopropanol, propylene glycol methyl ether, propylene glycol methyl ether acetate, diethylene glycol ethyl ether acetate, butanol, methyl isobutyl ketone, acetic acetate, ethyl lactate, butyl lactate, ethyl acetate, butyl acetate, diethylene glycol monoethylether acetate, diethylene glycol mono butyl ether acetate, propylene glycol diacetate, ethyl 3-ethoxy propionate, gamma-butylolactone, and mixtures thereof. In some preferred embodiments, the solvent to be purified includes, for example, propylene glycol methyl ether acetate; propylene glycol methyl ether; and mixtures thereof.

In one embodiment, the organic solvents useful in the present invention include, but are not limited to, for example, propylene glycol methyl ether, dipropylene glycol methyl ether, tripropylene glycol methyl ether, propylene glycol ethyl ether, propylene glycol propyl ether, ethylene glycol propyl ether, ethylene glycol butyl ether, diethylene glycol methyl ether, diethylene glycol ethyl ether, diethylene glycol propyl ether, diethylene glycol butyl ether, ethanol, isopropanol, and the like; and mixtures thereof.

In other embodiments, the organic solvents useful in the present invention include, but are not limited to, for example, “hydrolysable organic solvents”. As used herein, a “hydrolysable organic solvent” means a solvent including a compound which may be decomposed to acid and base components by water with or without a catalyst. Hydrolysable organic solvents include, but are not limited to, for example, esters, amides, carbonates, and mixtures thereof. Examples of esters useful in the present invention include propylene glycol methyl ether acetate (PGMEA), ethyl lactate, butyl lactate, ethyl acetate, butyl acetate, diethylene glycol monoethylether acetate, diethylene glycol mono butyl ether acetate, propylene glycol diacetate, ethyl 3-ethoxy propionate, gamma-butylolactone, and mixtures thereof.

In one preferred embodiment, the solvents to be purified (i.e., treated with the modified zeolite material) include, for example, glycol ethers, glycol ether acetates, alcohols, ketones, esters, and mixtures thereof.

As an illustration, and not to be limited thereby, some examples of solvents to be purified include: propylene glycol methyl ether, propylene glycol methyl ether acetate, diethylene glycol ethyl ether, butanol, methyl isobutyl ketone, acetic acetate, and the like; and mixtures thereof.

In some embodiments, various metal impurities can be present in the solvent before the solvent is purified using the modified zeolite material of the present invention, including, for example, Li, Na, K, Mg, Ca, Sr, Ba, Al, Cr, Mn, Fe, Co, Ni, Cu, Zn, Ag, Cd, Pb, Sb, and the like; and combinations thereof. In other embodiments, the particular contaminants desired to be removed from the solvent can include, for example, Na, K, Ca, Al, Fe, Ni, Zn, Cu, Sn, Cr and mixtures thereof. In one preferred embodiment, the metal impurities to be removed from the solvents include, for example, Ni, Cu, Sn, Cr, Ba, Cd, Pb, and mixtures thereof.

In one general embodiment, the targeting metal impurity level of an organic solvent, after treating the solvent with the above-described modified zeolite treatment, is in the ppb level or less. For example, in some embodiments, the targeting metal impurity level of an organic solvent, after treating the solvent with the above-described modified zeolite treatment, is less than 1,000 ppt (part per trillion). Therefore, the organic solvents obtained using the purifying process of the present invention can be useful in applications which requires a quite high level of pure solvent, such as for the manufacture of pharmaceuticals and electronic materials, and especially for use, for example, in semiconductor fabrication processes.

In other embodiments, the metal contaminants and other contaminants in the organic solvents processed through the modified zeolite process of the present invention can be, for example, from 0 ppt to less than 1,000 ppt in one general embodiment; from 1 to 800 ppt in another embodiment; from 1 to 500 ppt in still another embodiment; from 1 ppt to 400 ppt in yet another embodiment; from 1 ppt to 300 ppt in even still another embodiment; from 1 ppt to 200 ppt in even yet another embodiment; from 1 ppt to 100 ppt in another embodiment; and from 1 ppt to 50 ppt in still another embodiment. In other embodiments, some of the specific individual metal contaminants can be at a concentration level of less than 50 ppt. In some applications the total concentration of the contaminants in the solvent cannot exceed a certain concentration. For example, if the total contaminant level in the solvent is above 50 ppt and cannot be reduced to less than 50 ppt, then the solvent may not be useful for certain applications such as electronic processing.

Water may also be present in the solvent and the modified zeolite material can remove the water from the solvent to be purified. The target of water removal rate from the solvent is 80% or more in one embodiment; from 80% to 100% in another embodiment; from 85% to 98% in still another embodiment; and from 90% to 95% in yet another embodiment.

Sometimes a zeolite material originally provided for use in the modification process of the present invention may contain certain metal compounds originating from the manufacturing process of the zeolite material. Such metal compounds in the original (unmodified) zeolite material might leech out from the zeolite material and cause contamination in the solvent to be purified. Therefore, it is desirable to minimize the amount of metal compounds, leachable species, and/or other unwanted compounds such as water present in the zeolite material to reduce the potential for such contaminants to contaminate the solvent to be purified. To prevent metal contamination of a solvent where the contamination originates from the zeolite material, the contents of the metal compounds in the zeolite material to be used in one or more application embodiments of the present invention, are generally 20 wt % or less.

The original (unmodified) zeolite material before modification may also contain an undesired amount of water. In one general embodiment, the content of water in the zeolite material is decreased to 50 wt % or less, prior to use; and from 5 wt % to 30 wt % in another embodiment. To decrease the content of water in the zeolite material, the zeolite material can be regenerated before contacting the organic solvent. An apparatus of regeneration and conditions such as temperature, time and pressure for drying the zeolite material may be selected using techniques known to those of skill in the art. For example, the zeolite material can be heated at a temperature of from 250° C. to 300° C. for a period of time of from 0.5 hr to 16 hr under decompressed condition.

In some embodiments, the final metal content of a solvent to be used in various applications requiring a low metal content is less than 1 ppb which the zeolite material of the present invention can achieve. In some embodiments, the final water content of a solvent may depend on the application the solvent will be used. For various applications requiring a low water content, the zeolite material of the present invention can provide a solvent with a water content of 300 ppm or less in one general embodiment, 280 ppm or less in another embodiment, 200 ppm or less in still another embodiment, 100 ppm or less in yet another embodiment, 50 ppm or less in even still another embodiment, and 10 ppm or less in even yet another embodiment. In other embodiments, the zeolite material of the present invention can provide a water content for the solvent of from 0.01 ppm to 300 ppm in one general embodiment, and from 1 ppm to 280 ppm in another embodiment. In a preferred embodiment, the water content of the solvent can be from 1 ppm to 10 ppm in one general embodiment.

In broad embodiment, a process for removing metal contaminants from organic solvents, includes the steps of (a) providing a fixed bed of the modified zeolite described above; and (b) contacting an organic solvent or a mixture of organic solvents with the modified zeolite material in the fixed bed of step (a).

When contacting an organic solvent with a fixed bed of modified zeolite, any known conventional methods for contacting liquids with modified zeolite can be used. For example, a fixed bed of modified zeolite can be packed in a column and the solvent can be poured from the top of the column through the fixed bed of modified zeolite. In the contacting step (b) of the process, the flow rate of the solvent passing through the fixed modified zeolite bed can be, for example, from 0.1 BV/hr to 20 BV/hr (BV=bed volume) in one general embodiment; from 0.5 BV/hr to 10 BV/hr in another embodiment; and from 1 BV/hr to 5 BV/hr in still another embodiment. If the flow rate of the solvent passing through the fixed modified zeolite bed is above 20 BV/hr, the rate of metal removal will decrease; and if the flow rate of the solvent passing through the fixed modified zeolite bed is below 0.1 BV/hr, the purification efficiency of the solvent will decrease. As used herein, “BV” means bed volume, and refers to an amount of liquid contacted with the same amount of a fixed bed of modified zeolite. For example, if 120 mL of a fixed bed of modified zeolite is used, 1 BV means 120 mL of organic solvent is contacted with the fixed bed of modified zeolite. “BV/hr” is calculated by flow rate (mL/hr) divided by bed volume (mL).

In general, the temperature step (b) of the process, i.e., contacting an organic solvent with a fixed bed of modified zeolite material can include, for example, from 0° C. to 100° C. in one embodiment, from 10° C. to 60° C. in another embodiment, and from 20° C. to 40° C. in still another embodiment. If the temperature is above 100° C., the modified zeolite will be damaged; and if the temperature is below 0° C., some of the solvents to be treated may freeze.

In general, the resultant purified organic solvent processed through a zeolite material of the present invention has at least an 80% improvement, i.e., at least an 80% reduction of the amount of contaminants from the original amount of contaminants present in the original solvent containing metal contaminants. For example, the reduction of contaminants in the solvent can be from ≥80% to 100% in one general embodiment; from 90% to 100% in another embodiment; and from 95% to 99% in still another embodiment. Alternatively, for example, as aforementioned, in some embodiments, the metal contaminants and other contaminants in the organic solvents processed through the modified zeolite process of the present invention can be removed and reduced to a level measured in the less than ppb level; and in a preferred embodiment the contaminants can be removed and reduced to a level measured in the less than ppt level. For example, as described above, the metal contaminant content of the solvent can be reduced to a level of 0 ppt to less than 1,000 ppt in one general embodiment; and other embodiments previously described above.

In addition, the resultant purified organic solvent processed through a zeolite material of the present invention has at least an 80% improvement, i.e., at least an 80% reduction of the water content from the original amount of water content present in the original solvent containing water. Alternatively, for example, as aforementioned, in some embodiments, the water content in the solvent using the modified zeolite material of the present invention can be reduced the water content in the solvent by 80% or more in one general embodiment; from ≥80% to 100% in another embodiment; from 90% to 100% in still another embodiment; and from 95% to 99% in yet another embodiment.

In some embodiments, the purified solvent of the present invention is used, for example, in display pixel processing applications; in display thin film transistor processing applications, and in semiconductor circuit processing applications.

### Examples

The following Inventive Examples (Inv. Ex.) and Comparative Examples (Comp. Ex.) (collectively, “the Examples”) are presented herein to further illustrate the features of the present invention but are not intended to be construed, either explicitly or by implication, as limiting the scope of the claims. The Inventive Examples of the present invention are identified by Arabic numerals and the Comparative Examples are represented by letters of the alphabet. The following experiments analyze the performance of embodiments of compositions described herein. Unless otherwise stated all parts and percentages are by weight on a total weight basis.

### Designations

Various terms and designations used in the Inventive Examples (Inv. Ex.) and the Comparative Examples (Comp. Ex.) are explained as follows:
“BV” stands for bed volume.
“HDPE” stands for high density polyethylene.
“PTFE” stands for polytetrafluoroethylene.
“PFA” stands for polyfluoroalkoxy.

### Raw Materials

Table I describes the raw materials (ingredients) including the solvent and the different zeolite materials used in the Examples. The solvent to purify and used in the Examples was a glycol ether acetate, and more specifically, a propylene glycol methyl ether acetate (e.g., DOWANOL™ PMA).

TABLE I
Ingredients

| | Chemical Description, Chemical | |
|---|---|---|
| Product (Ingredient) | Formula, or Structure (Pore Size) | Source |
| Zeolite A | Al₂O₃•2SiO₂•5H₂O; (4 Å) | Tosoh Corporation; and subsequently |
| | | modified as described below. |
| Zeolite B | (NH₄)₂O•Al₂O₃•2SiO₂•4H₂O; (4 Å) | Tosoh Corporation; and subsequently |
| | | modified as described below |
| Zeolite C | Na₂O•Al₂O₃•2SiO₂•4H₂O; (4 Å) | Tosoh Corporation; and subsequently |
| | | modified as described below. |
| Zeolite D | Al₂O₃•2SiO₂•5H₂O; (10 Å) | Tosoh Corporation; and subsequently |
| | | modified as described below. |
| Zeolite E | (NH₄)₂O•Al₂O₃•2SiO₂•4H₂O; (10 Å) | Tosoh Corporation; and subsequently |
| | | modified as described below. |
| DOWANOL ™ | Solvent; propylene glycol methyl | The Dow Chemical Company (DOW) |
| PMA | ether acetate (PGMEA) | |

TABLE II
Zeolite Characteristics

| Zeolite: | Zeolite A | Zeolite B | Zeolite C | Zeolite D | Zeolite E |
|---|---|---|---|---|---|
| Type: | Hydrogen | Ammonium | Sodium | Hydrogen | Ammonium |
| Shape: | Bead | Bead | Bar | Bead | Bead |
| Average | 4 Å | 4 Å | 4 Å | 10 Å | 10 Å |
| Pore size: | | | | | |
| Structure: | Al₂O₃•2SiO₂•5H₂O | (NH₄)₂O•Al₂O₃•2SiO₂•4H₂O | Na₂O•Al₂O₃•2SiO₂•4H₂O | Al₂O₃•2SiO₂•5H₂O | (NH₄)₂O•Al₂O₃•2SiO₂•4H₂O |

### General Procedure for Zeolite Modification

In one part of the procedure, a hydrogen-based zeolite was prepared by first placing 50 mL of unmodified zeolite having a pore size of 4 Å to 10 Å as a fixed bed in a column of a diameter of 5 cm and a length of 30 cm. The unmodified zeolite was modified by flushing acetic acid through the fixed bed column at 2 BV/hr of flow rate for 1 day. The flushing step was controlled at a temperature of 120° C. while applying nitrogen gas. After 180 min of the above flushing step, the resultant 4 Å-10 Å zeolite was changed to a hydrogen-based zeolite (e.g., Zeolite A and Zeolite D).

In another part of the procedure, an ammonia-based zeolite was prepared by first placing 50 mL of unmodified zeolite having a pore size of 4-10 Å as a fixed bed in a column of a diameter of 5 cm and a length of 30 cm. The unmodified zeolite was modified by flushing 1 mol % (NH₄)₂SO₄ aqueous solution at 2 BV/Hr of flow rate for 1 day. The flushing step was controlled at a temperature of at 120° C. while applying nitrogen gas. After 180 min of the above flushing step, the resultant 4-10 Å zeolite was changed to an ammonia-based Zeolite (e.g., Zeolite B and Zeolite E).

In still another part of the general procedure, a modified zeolite material useful in the present invention was prepared by mixing: (i) the hydrogen-based zeolite (Zeolite A) prepared as described above and (ii) the ammonia-based zeolite (Zeolite B) prepared as described above, wherein the two zeolite materials were mixed together in a Zeolite A:Zeolite B volume ratio of 1:1.

The above modified zeolite material comprising the mixture of the hydrogen-based zeolite and the ammonia-based zeolite was activated through calcinating the mixed zeolite at a temperature of 250° C. for 8 hr in a nitrogen atmosphere (0.1 MPa) to carry out dehydration.

### Dehydration and Metal Exchange

A 100-mL mixture of Zeolite A and Zeolite B (1:1 volume ratio) was placed at bottom of column. Then, 50 mL of Zeolite C was placed on top of the mixture of Zeolite A and Zeolite B. The Comparative Example (Comp. Ex. A) used a column with Zeolite C only.

A solvent, propylene glycol methyl ether acetate, was passed through the zeolite bed at 2 BV/Hr of flow rate. A nitrogen (N₂) padding was applied to the inside of the column with 0.1 MPa N₂ pressure. The operation of passing the solvent through the zeolite bed performs a dehydration and metal exchange function. The step of passing the solvent through the zeolite bed was conducted at a temperature controlled at 80° C.

A sample of solvent passing through the zeolite bed was collected in a 100 mL plastic (HDPE) bottle at various periods of time during the above dehydration and metal exchange process. A first 100 mL sample of solvent was collected at 0 hr followed by subsequent collections of solvent samples of certain Examples at 2 hr, 4 hr, 6 hr, and/or 8 hr.

TABLE III
Zeolite Compositions and Solvent

| | Example No.: | | | | | | | | | | | | | | | | | |
|---|---|---|---|---|---|---|---|---|---|---|---|---|---|---|---|---|---|---|
| | Comp. Ex. A | | | Inv. Ex. 1 | | | | Inv. Ex. 2 | | | | Inv. Ex. 3 | | | Inv. Ex. 4 | | | |
| Time of Sample: | 0 hr | 2 hr | 6 hr | 0 hr | 2 hr | 4 hr | 8 hr | 0 hr | 2 hr | 4 hr | 6 hr | 0 hr | 4 hr | 6 hr | 0 hr | 2 hr | 4 hr | 6 hr |
| DOWANOL ™ | 100 | 100 | 100 | 100 | 100 | 100 | 100 | 100 | 100 | 100 | 100 | 100 | 100 | 100 | 100 | 100 | 100 | 100 |
| PMA (wt %): | | | | | | | | | | | | | | | | | | |
| Zeolite A (vol %): | | | | | 50 | 50 | 50 | | 25 | 25 | 25 | | | | | | | |
| Zeolite B (vol %): | | | | | 50 | 50 | 50 | | 25 | 25 | 25 | | | | | | | |
| Zeolite C (vol %): | | 100 | 100 | | | | | | 50 | 50 | 50 | | 50 | 50 | | | | |
| Zeolite D (vol %): | | | | | | | | | | | | | 25 | 25 | | 50 | 50 | 50 |
| Zeolite E (vol %): | | | | | | | | | | | | | 25 | 25 | | 50 | 50 | 50 |

### Test Methods and Measurements

The column and pipelines used in the Examples were fabricated from fluoroplastic (e.g., PTFE or PFA) or electropolished stainless steel.

### Metal Concentration

The metal content of the solvent samples processed through the above dehydration and metal exchange process was measured and analyzed by conventional equipment such as a ICP-MS (Inductively Coupled Plasma-Mass Spectrometry) instrument available from Rion following the metal testing method using the ICP-MS; and the analytical results are described in Table IV-Table VI.

### Water Test

The water content of the solvent processed through the above dehydration and metal exchange process was measured by Karl Fischer titration using the water test method described in ASTM E203.

### Results of Tests

The metal contamination removal rate can be determined by comparing the data of 2 hr, 4 hr, or 6 hr (after zeolite treatment) comparted to the data of 0 hr (before zeolite treatment) as described in Table IV-Table VI. In general, over a period of time, the metal content in a solvent sample will increase or decrease; and eventually, the metal content achieves an equilibrium level.

TABLE IV
Properties/Performance (Comp. Ex. A and Inv. Ex. 1)

| | Comp. Ex. A | Comp. Ex. A | Comp. Ex. A | Inv. Ex. 1 | Inv. Ex. 1 | Inv. Ex. 1 |
|---|---|---|---|---|---|---|
| Property | @ 0 hr | @ 2 hr | @ 6 hr | @ 0 hr | (@ 2 hr | @ 4 hr |
| Water content (ppm): | 609 | 627 | 388 | 500 | 351 | 379 |
| Ni (ppb): | 0.07 | 0.20 | 0.14 | 0.01 | 0.01 | 0.01 |
| Ni removal rate (%): | — | increase | increase | — | — | — |
| Cu (ppb): | 0.37 | 1.17 | 0.38 | 0.01 | 0.01 | 0.01 |
| Cu removal rate (%): | | increase | increase | — | — | — |
| Cr (ppb): | 0.06 | 0.25 | 0.33 | 0.05 | 0.03 | 0.04 |
| Cr removal rate (%): | — | increase | increase | — | 40.0 | 20.0 |
| Sn (ppb): | 2.22 | 2.34 | 5.30 | 2.36 | 0.79 | 0.70 |
| Sn removal rate (%): | — | increase | increase | — | 66.5 | 70.3 |
| Ba (ppb): | 0.11 | 0.12 | 0.15 | 0.03 | 0.01 | 0.01 |
| Ba removal rate (%): | — | increase | increase | — | 66.7 | 66.7 |
| Pb (ppb): | 0.10 | 0.14 | 0.06 | 0.00 | 0.00 | 0.00 |
| Pb removal rate (%): | — | increase | 40.0 | — | — | — |
| Na (ppb): | 1.57 | 5.70 | 6.10 | 0.14 | 0.40 | 0.19 |
| Na removal rate (%): | — | increase | increase | — | increase | increase |

TABLE V
Properties/Performance (Inv. Ex. 2)

| | Inv. Ex. 2 | Inv. Ex. 2 | Inv. Ex. 2 | Inv. Ex. 2 |
|---|---|---|---|---|
| Property | @ 0 hr | @ 2 hr | @ 4 hr | @ 6 hr |
| Water content (ppm): | 434 | 413 | 451 | 487 |
| Ni (ppb): | 0.08 | 0.06 | 0.05 | 0.06 |
| Ni removal rate (%): | — | 25.0 | 37.5 | 25.0 |
| Cu (ppb): | 0.40 | 0.16 | 0.13 | 0.12 |
| Cu removal rate (%): | — | 60.0 | 67.5 | 70.0 |
| Cr (ppb): | 0.05 | 0.09 | 0.09 | 0.10 |
| Cr removal rate (%): | — | increase | increase | increase |
| Sn (ppb): | 2.25 | 2.13 | 1.46 | 1.54 |
| Sn removal rate (%): | — | 5.3 | 35.1 | 31.6 |
| Ba (ppb): | 0.05 | 0.06 | 0.03 | 0.04 |
| Ba removal rate (%): | — | increase | 40.0 | 20.0 |
| Pb (ppb): | 0.17 | 0.17 | 0.09 | 0.09 |
| Pb removal rate (%): | — | — | 47.1 | 47.1 |
| Na (ppb): | 1.40 | 1.53 | 1.28 | 1.44 |
| Na removal rate (%): | — | increase | 8.6 | increase |

TABLE VI
Properties/Performance (Inv. Ex. 3 and Inv. Ex. 4)

| | Inv. Ex. 3 | Inv. Ex. 3 | Inv. Ex. 3 | Inv. Ex. 4 | Inv. Ex. 4 | Inv. Ex. 4 | Inv. Ex. 4 |
|---|---|---|---|---|---|---|---|
| Property | @ 0 hr | @ 4 hr | @ 6 hr | @ 0 hr | @ 2 hr | @ 4 hr | @ 6 hr |
| Water content (ppm): | 508 | 320 | 470 | 738 | 310 | 298 | 277 |
| Ni (ppb): | 0.08 | 0.07 | 0.05 | 0.05 | 0.03 | 0.01 | 0.01 |
| Ni removal rate (%): | — | 12.5 | 37.5 | — | 40.0 | 80.0 | 80.0 |
| Cu (ppb): | 0.42 | 0.19 | 0.18 | 0.11 | 0.10 | 0.08 | 0.07 |
| Cu removal rate (%): | — | 54.8 | 57.1 | — | 11.1 | 27.2 | 36.4 |
| Cr (ppb): | 0.08 | 0.05 | 0.04 | 0.05 | 0.07 | 0.03 | 0.01 |
| Cr removal rate (%): | — | 37.5 | 50.0 | — | increase | 40.0 | 80.0 |
| Sn (ppb): | 2.43 | 1.10 | 0.24 | 1.41 | 0.49 | 0.13 | 0.10 |
| Sn removal rate (%): | — | 54.7 | 90.1 | — | 57.0 | 90.8 | 92.9 |
| Ba (ppb): | 0.03 | 0.08 | 0.04 | 0.02 | 0.02 | 0.00 | 0.00 |
| Ba removal rate (%): | — | increase | increase | — | — | 100.0 | 100.0 |
| Pb (ppb): | 0.10 | 0.04 | 0.01 | 0.04 | 0.02 | 0.00 | 0.00 |
| Pb removal rate (%): | — | 60.0 | 90.0 | — | 50.0 | 100.0 | 100.0 |
| Na (ppb): | 0.58 | 0.76 | 0.61 | 4.29 | 4.00 | 0.60 | 0.41 |
| Na removal rate (%): | — | increase | increase | — | 6.8 | 86.0 | 90.4 |

### Discussion of Results

Comp. Ex. A: A commercially available sodium-based zeolite with a 4 Å pore size was used in Comp. Ex. A. The sodium-based zeolite removed water from a solvent sample, but could not remove most metals from the solvent sample except Pb purified at 6 hr (at a 40% removal rate). Instead, the sodium-based zeolite would capture metals from treating previous material and release to following material which cause an incremental increase of metal content, especially for Na, where the metal content increased significantly.
Inv. Ex. 1: A half volume of a hydrogen-based zeolite with a 4 Å pore size and the other half volume of an ammonium-based zeolite with a 4 Å pore size were used in Inv. Ex. 1. This combination removed Cr, Sn, and Ba in PMA after treatment. The Sn removal rate at 4 hr showed over 70% efficiency. The results show that the modified zeolite of the present invention advantageously and effectively removes transition metals from a solvent.
Inv. Ex. 2: A half volume of the zeolite combination material of Inv. Ex. 1 and a half volume of the sodium-based zeolite of Comp. Ex. A was used in Inv. Ex. 2. The top layer of the zeolite material in the column was the zeolite of Comp. Ex. A; and the bottom layer of the zeolite material in the column was the zeolite of Inv. Ex. 1. The zeolite material in this Inv. Ex. 2 removed most metals except Cr and Na. The Cu removal rate at 6 hr showed a 70% efficiency. The results show that the modified zeolite of the present invention advantageously and effectively removes transition metals from a solvent.
Inv. Ex. 3: A half volume of the zeolite material of Inv. Ex. 4 (see Inv. Ex. 4 below) and a half volume of the sodium-based zeolite of Comp. Ex. A was used in Inv. Ex. 3. The top layer of the zeolite material in the column was the zeolite of Comp. Ex. A; and bottom layer of the zeolite material in the column was the zeolite of Inv. Ex. 4. The zeolite material in this Inv. Ex. 3 removed most metals from a solvent sample except Ba and Na. The Sn removal rate at 6 hr and the Pb removal rate at 6 hr showed no less than 90% efficiency. The results also show that the modified zeolite of the present invention (as a layer-by-layer design) removes a small portion of water from a solvent. The advantage of the zeolite material of this Inv. Ex. 3 is the effectiveness of removing some transition metals from a solvent.
Inv. Ex. 4: A half volume of a hydrogen-based zeolite material with a 10 Å pore size and the other half of an ammonia-based zeolite material with a 10 Å pore size were used in Inv. Ex. 4. The combination of zeolite materials of this Inv. Ex. 4 removed all metals and most metal removal rates reached an efficiency of 80% or above. In addition, water reduction was good versus CE1. The advantage of the zeolite material of this Inv. Ex. 4 is the effectiveness of removing all metals and water.

The data of the Comp. Ex. A shows metal increasing during the solvent purification process. It is theorized that metal increase was caused by the metals captured from previous processed solvent material were subsequently released into subsequent processed solvent material following the previous processed solvent material. The zeolite material of Comp. Ex. A shows a weak metal exchange strength. In contrast, the majority of the zeolite materials of the Inv. Ex. 1-4, showed that the metal content of a solvent can be reduced and showed a strong metal exchange strength. A metal removal rate of over 70% exhibited by a zeolite material of the present invention is considered an efficient performance. With regard to pore size, using a zeolite material with a 10 Å pore size is more efficient than using a zeolite material with a 4 Å pore size in terms of metal removal and water removal.

Although the results show that the zeolite materials of Inv. Ex. 2 and Inv. Ex. 3 were less effective than Comp. Ex. A in terms of water removal, the zeolite materials of Inv. Ex. 2 and Inv. Ex. 3 illustrate the effectiveness of the zeolite materials of the present invention even when mixed with a sodium-based zeolite with 4 Å pore size in terms of metal removal. The combination of a hydrogen-based zeolite and an ammonia-based zeolite used in Inv. Ex. 1 and Inv. Ex. 4 were significantly better than Comp. Ex. A in terms of metal removal rate.

What is claimed is:

1. A modified zeolite comprising a zeolite material for purifying a solvent; wherein the zeolite material is selected from the group consisting of (i) a hydrogen-based zeolite having hydrogen functionalities, (ii) an ammonium-based zeolite having ammonium functionalities, and (iii) a combination of a hydrogen-based zeolite having hydrogen functionalities and an ammonium-based zeolite having ammonium functionalities; wherein the zeolite contains a pore size of from 3 Å to 20 Å to selectively adsorb metal impurities and water present in the solvent.

2. The modified zeolite of claim 1, wherein the zeolite includes an aluminosilicate framework including aluminum, silicon, oxygen, and counter cations; and a honeycomb structure.

3. The modified zeolite of claim 1, wherein the zeolite has a molecular structure of M₂O·xAl₂O₃·ySiO₂·zH₂O; and wherein M is at least one of H, NH₄, NRH₃, NR₂H₂, NR₃H, NR₄ or metal oxides; R is methyl; x is from 0.2 to 5; y is from 1 to 25 and z is from 1 to 20.

4. The modified zeolite of claim 1, wherein the zeolite has a surface area of from 500 m²/g to 5,000 m²/g; a cation exchange capacity of from 0.1 eq/mol to 1.5 eq/mol; and a stability of pH value of liquid from 0 to 12.

5. The modified zeolite of claim 1, wherein the zeolite material is prepared by an alkali metal reduction process; and wherein the zeolite material is an alkaline-based zeolite, an alkali-based zeolite, a hydrogen-based zeolite, or mixtures thereof.

6. A process for preparing the modified zeolite of claim 1 comprising the steps of:
(a) placing an unmodified zeolite material in a column to form a fixed bed of unmodified zeolite material;
(b) modifying the fixed bed of unmodified zeolite material from step (a) to form a modified zeolite material by: (i) flushing at least one weak acid through the fixed bed column of step (a) for forming a hydrogen-based zeolite; or (ii) flushing at least one weak base or at least one ammonia salt through the fixed bed column of step (a) for forming an ammonium-based zeolite; and
(c) activating the modified zeolite material from step (b) by calcinating the zeolite material at a temperature of from 200° C. to 400° C. to dehydrate the zeolite material.

7. A process for removing metal contaminants from a contaminated organic solvent or a mixture of contaminated organic solvents comprising the steps of:
(a) placing an unmodified zeolite material in a column to form a fixed bed of unmodified zeolite material;
(b) modifying the fixed bed of unmodified zeolite material from step (a) by flushing a weak acid to form a hydrogen-based zeolite material; or flushing a weak base or ammonia salt to form an ammonium-based zeolite material through the fixed bed column of step (a) to form a modified zeolite material; and
(c) activating the modified zeolite material from step (b) by calcinating the zeolite material at a temperature of from 200° C. to 400° C. to substantially dehydrate the zeolite material and form a modified zeolite material;
(d) providing a fixed bed of the modified zeolite material from step (c); and
(e) contacting a metal-contaminated organic solvent or a mixture of metal-contaminated organic solvents to be purified with the modified zeolite material in the fixed bed from step (d) to form a purified solvent having a metal content of less than 1 parts per billion.

8. The process of claim 7, wherein the weak acid in the flushing step (b) is acetic acid, propanoic acid, or mixtures thereof; or wherein the weak base in the flushing step (b) is ammonia; wherein the flow rate of the flushing step (b) is from 0.1 BV/hr to 20 BV/hr; wherein the flushing step (b) is conducted at a temperature of from room temperature to 100° C.; and wherein the flushing step (b) is conducted in an inert gas atmosphere at an inert gas pressure of from 0.1 MPa to 0.3 MPa; and wherein the inert gas is nitrogen, argon, or mixtures thereof.

9. The process of claim 7, wherein the solvent to be purified is selected from the group consisting of alcohols, ethers, ketones, acetates, hydrocarbons, halogenated compounds, esters, and mixtures thereof.

10. The process of claim 7, wherein the solvent to be purified is a glycol ether or a glycol ether acetate.

11. The process of claim, wherein the solvent to be purified is propylene glycol methyl ether acetate.

12. The process of claim 7, wherein the solvent to be purified has a metal impurities content of greater than 1 part per billion; and wherein the solvent to be purified is contacted with the modified zeolite in step (e) to form a purified solvent containing a metal impurities content of less than 1 part per billion; and the water content of the solvent is reduced to less than 0.1 weight percent.

13. A purified solvent prepared by the process of claim 7.

14. The purified solvent of claim 13, wherein the concentration of the individual metal impurities present in the purified solvent is less than 100 parts per trillion.
